(19) United States
(12) Patent Application Publication
(10) Pub. No.: US 2021/0233620 A1
(43) Pub. Date: Jul. 29, 2021

(54) MACHINE LEARNING

(71) Applicant: THE UNIVERSITY COURT OF THE UNIVERSITY OF GLASGOW, Glasgow, Strathclyde (GB)

(72) Inventor: Leroy CRONIN, Glasgow Strathclyde (GB)

(21) Appl. No.: 17/257,227

(22) PCT Filed: Jul. 4, 2019
(86) PCT No.: PCT/EP2019/067948

§ 371 Date: Dec. 30, 2020

(30) Foreign Application Priority Data
Jul. 4, 2018 (GB) 1810944.7

(51) Int. Cl. G16C 20/70; G16C 20/10; G01J 3/10

(52) U.S. Cl. CPC G16C 20/70; G01J 3/108; G16C 20/10

(57) ABSTRACT

The present invention provides a method to generate a predictive model for a reaction set, which reaction set is the sum of the reaction outcomes for a plurality of chemical inputs. Also provided is a system for generating a predictive model for a reaction set, which system may be used in the method. The system comprises a synthesiser for conducting reactions, which synthesiser is an automated synthesiser, an analytical unit for monitoring reactions performed by the synthesiser, and a control unit suitably programmed with a machine learning algorithm, for analysing analytical data from the analytical unit, and for controlling the synthesiser.

### RELATED APPLICATION

The present case claims the benefit of, and priority, to GB 1810944.7 filed on 4 Jul. 2018 (4.07.2018), the contents of which are hereby incorporated by reference in their entirety.

### FIELD OF THE INVENTION

The present invention provides a system for performing chemical reactions, and a method or using the system to perform explore the reactivity of a given set of chemical and physical inputs using machine learning. Also provided is a method for predicting the outcome of a reaction using the system of the invention.

### BACKGROUND

Exploring the reactivity of different compounds under a range of conditions is a challenge but it is a useful way to discover new reactions, molecules, and methodologies (Reymond et al.) This means that expert chemists are constantly aiming to explore new combinations of reagents, catalysts, solvents, and process conditions (for example, order of reagent addition, reaction temperature, and so on) looking for new reactivity, reactions, and molecules (Allen et al.; Naredla et al.) Also, the fact that reactivity can also be altered by reaction process conditions and catalysts, makes the discovery process inherently unpredictable. An attractive alternative is to instead predict the reactivity of organic molecules, but most of these approaches are still in their infancy (Warr et al.) This is because reaction prediction using high-level quantum chemistry methods, even for simple molecules is fundamentally difficult, owing to the need to calculate an accurate potential energy surface for all the possible reaction pathways (see Plata et al.) Nevertheless, chemists are able to identify reactivity patterns and formulate chemical hypotheses using their intuition about the regions of chemical space that might yield discovery, and then do experiments in an iterative fashion learning from previous experiments (see Graulich et al.)

Some researchers are using existing databases of chemical and biological reactivity, to build predictive models, but these models are frequently not very accurate, as the data recorded in the database is not always correct, and importantly failed experiments are not captured.

The present inventor has previously described in WO 2013/175240 a system and a method for exploring a reaction space using a genetic algorithm. Here, the genetic algorithm operates to find within the reaction space a product or a reaction having a characteristic that that meets or exceeds a specification set by a user. The genetic algorithm allows the space to be explored without reverting to the performance of all possible reactions within the reaction space.

In this earlier work, the genetic algorithm is capable of linking positive chemical and physical inputs with positive results, but it cannot accurately describe the relationship between various inputs, nor can it accurately predict a particular result for a combination of inputs.

In particular, the genetic algorithm requires the performance of reactions to identify those meeting or exceeding the specification. The system cannot predict which combinations of inputs will meet or exceed the specification in the absence of the reaction performance.

There is a need for a system for reaction prediction and methods for reaction prediction.

### SUMMARY OF THE INVENTION

In a general, the present invention provides a system for use in reaction prediction using machine learning, and methods of synthesis by machine learning for the purpose of reaction prediction. The present invention provides an integrated reaction system whereby chemical inputs, a synthesiser, and reaction outputs can be programmed, and the reactivity assessed in real time, for example using NMR spectroscopy, IR spectroscopy, and mass spectrometry, amongst others.

The system and methods of the invention may be used to generate a predictive model for a reaction set, which is a collection of interrelated reactions having a common reaction element, such as a common reagent or catalyst, or a common reaction mechanism. The systems and methods explore the reactivity within the available reaction space, and are permitted to investigate successful and unsuccessful reaction outcomes in order to generate the predictive model.

The system for use in the invention comprises a synthesiser for conducting reactions, which synthesiser is an automated synthesiser, an analytical unit for monitoring reactions performed by the synthesiser, and a control unit suitably programmed with a machine learning algorithm, for analysing analytical data from the analytical unit, and for controlling the synthesiser.

The present invention shows that a system having an automated synthesiser, such as a chemical robot, together with an analytical unit and a control unit, under the control of a machine learning algorithm, can be used to autonomously search organic chemical space for new reactivity, leading to the discovery of new reactions and new products.

By exploiting the machine learning algorithms, the system can navigate between reactive and non-reactive mixtures autonomously, learning reactivity patterns, and efficiently explore the most reactive regions of chemical space, requiring no prior knowledge. Furthermore, after training, the system needs to perform only a fraction of possible the reactions to explore the most reactive/interesting parts of the chemical space. This leads to a much better performance than the random high throughput screening methods, saving time and resources.

The in silico analysis of analytical data for reactions reported in the art shows that the methods of the invention can be easily expanded to search complex chemical systems including important transition metal catalyzed transformations, modifying more reactions parameters such as reaction catalysts, ligands, additives, temperature and solvent, which maximizes the capability of this system.

In one aspect of the invention there is provided a method for developing a predictive model for a reaction set, the method comprising the steps of:
(i) providing a system according to the invention, the system comprising a synthesiser for conducting reactions, which synthesiser is an automated synthesiser, an analytical unit for monitoring reactions performed by the synthesiser, and a control unit suitably programmed with a machine learning algorithm, for analysing analytical data from the analytical unit, and for controlling the synthesiser
(ii) making available to the synthesiser chemical inputs, optionally together with physical inputs;
(iii) permitting the synthesiser to perform a series of reactions using the available chemical inputs, optionally together with physical inputs, wherein the series is a subset of all the possible reactions for the combinations of the available chemical inputs, optionally together with physical inputs;
(iv) permitting the analytical unit to analyse each reaction, and allowing the analytical unit to transmit analytical data to the control unit;
(v) allowing the control unit to consider the analytical data to determine a reaction outcome for each reaction, and considering the reaction outcomes in association with the chemical inputs, optionally together with the physical inputs, for each reaction; and
(vi) developing a predictive model using the machine learning algorithm for the reaction set from the subset of reactions.

The methods of the present case represent a development of intelligent automated approaches to chemical and biological discovery driven by machine learning systems, trained by human experts in a bottom-up approach, in contrast to the top-down fragment-based approach of the traditional chemist and biologist (see Palazzolo et al.)

These and other aspects and embodiment of the invention are described in further detail below.

### SUMMARY OF THE FIGURES

FIG. 1 shows a schematic of the chemical-robot for use in a system according to an embodiment of the invention, where the circles are pumps, and the coloured dots are the valves positions.

FIG. 2 shows (a) a SVM workflow for reaction detection using IR and NMR spectroscopy; (b) shows an example of ¹H NMR (43 MHz, MeCN) spectrum for an exemplary non-reactive reaction mixture; (c) shows an example of a reaction mixture ¹H NMR (43 MHz, MeCN) spectrum for which an exemplary chemical reaction has been detected; and (d) shows the available reaction space representation using vectors.

FIG. 3 is a schematic overview of a system for exploration of chemical space working with the liquid handling robot according to an embodiment of the invention, where the liquid handling robot for performing reactions chooses reactants from the a pool of starting materials, and the system is provided with an analytical unit for real time analysis of reaction outcomes, which can be rated as reactive or non-reactive, a control until operating under a machine learning algorithm, which is a type of artificial intelligence algorithm, for building a model of chemical space using machine learning and for recommending the next experiments, and controlling the robot, and a reaction database for storing reaction outcomes.

FIG. 4 shows the chemical inputs (1-18) used in a platform used to search for new transformations and to evaluate the performance of a system according to an embodiment of the invention.

FIG. 5 shows the simulations for exploring a chemical space and the predictive power of a model according to an embodiment of the invention, where (a) is the LDA projection of all the reactions performed, demonstrating the predictive power of LDA in classifying the reactivity; Red dot—reactive combination, blue dot—unreactive combination. Examples of reactions in different regions of chemical space: very reactive; moderately reactive; non-reactive; combinations of starting materials as projected by LDA based on collective chemical knowledge gathered by the robot; (b) is the simulation showing the average accuracy of LDA vs the percent of the space being explored 100 times with confidence intervals; and (c) is the simulation showing the number of reactive and unreactive mixtures done by the robot as space is being explored.

FIG. 6 shows (a) the reaction space of Suzuki-Miyaura reaction: identity of reactants, ligand, base and solvent and its vector representation for machine learning; (b) shows a validation of the predictive power of a model according to an embodiment of the invention for the test set of 30% of the reactions (1,728 reactions); and (c) shows a simulation of the machine learning controlled exploration of this reaction space. The yellow bar show initial random choice of 10% of reaction space (576 reactions) which had average yield of 39% and standard deviation (SD) of 27%. The green bars show the next batches as chosen by ML algorithm; for example the first batch of 100 reaction had mean yield of 85% with SD=14%.

FIG. 7 shows the multicomponent reactions discovered with a ML-driven robot according to an embodiment of the invention, where (a) is multicomponent reactions discovered between methyl propiolate (16), benzofuroxan (7), and DBU (13); (b) shows the ¹H NMR spectrum recorded for this reaction in the platform (red) and theoretical spectrum being sum of the starting materials; (c) shows the suggested mechanism of this multicomponent transformation; (d) shows a small library of compounds synthesized using the discovered reaction; (e) shows a multicomponent reaction of DMAP (12), DMAD (1) and nitrobenzene (14) leading to the derivative of 2,5-dihydrofuran 24; and (f) shows the solidi state structure of compound cis-24 (at 50% probability level).

FIG. 8 shows the novel reactivity discovered with a ML-driven robot according to an embodiment of the invention, where (a) shows the synthesis of chlorocyaninitrone (25) from nitrosobenzene (14) and trichloroacetonitrile (5) in the presence of DBU (13), and the X-ray structure of compound (25); (b) shows the ¹H NMR spectrum registered in the robotic platform for this transformation; (c) shows the novel reactivity of phenylketene with DBU; (d) shows the ACPI-MS spectrum registered in the robotic platform for the reaction of phenylacetylchloride with DBU (13); (e) shows a plausible mechanism for reaction of phenylketene with DBU (13); and (f) shows a possible mechanism for the formation of chlorocyanonitrone (25).

FIG. 9 shows (a) the Tanimoto similarity measure for the discovered reactions in the present case against the 3.5 million known reactions, measuring the difference in structure between the product and starting materials; and (b) the statistics for the discovered reactions.

FIG. 10 shows a flow diagram for a method according to an embodiment of the invention for the exploration of a chemical space in a reaction set.

FIG. 11 shows a schematic of a neural network used for yield prediction in a method according to an embodiment of the invention.

FIG. 12 shows (a) a loss for training and validation sets during training; and (b) the correlation for test sets between predicted and experimental yield for the test set.

FIG. 13 shows (a) a closed loop exploration of chemical space of Suzuki-Miyaura reaction searching for maximum yield; and (b) the average yields and standard deviation of the yield during exploration of the chemical space. The left-hand bar represents the random search containing 10% of chemical space (576 reactions) and the remaining bars show subsequent batches of 100 reaction chosen by neural network.

### DETAILED DESCRIPTION

Inspired by the creativity and intuition of the chemist, the inventors have found that a reaction system having an automated synthesiser and a control unit operated by a machine learning algorithm can explore more reactions than a bench chemist or biologist, and can do so quickly, particularly if the system is trained by an expert (see, for example Gil et al.)

A robotic approach to chemistry also gives information about failed or non-reactive experiments. The importance of negative results has been recently demonstrated for finding boundary conditions for crystallization of templated vanadium selenites (see, for example, Raccuglia et al.)

The system of the invention is suitable for autonomous reactivity searching of organic reagents. Here, the system can perform reactions, such as chemical reactions, automatically. The outcome of each reaction can be evaluated using proper analytical techniques and sensors. The system has a control unit under the control of an algorithm to efficiently navigate the chemical space defined by the set of input reagents. Recent progress in automated chemistry, on-line analytics, and real-time optimization have allowed the present inventors to construct a system, such as a chemical robot, for autonomously exploring chemical reactions (see also Sans et al.)

The system of the present invention comprises a reaction unit for conducting reactions, which reaction unit is an automated synthesiser, an analytical unit for monitoring reactions, and a control unit suitably programmed with a machine learning algorithm, for analysing analytical data from the analytical unit, and for controlling the automated synthesiser.

The inventor has previously described in WO 2013/175240 a system and a method for exploring an available chemical reaction space. The system and methods here allow for the identification of products and methods having a desired property. However, the methods developed there did not allow the user to predict a particular outcome for a reaction of interest, particularly as the methods focused only on those reactions that were seen to give the desirable result, without any great understanding about why that results were achieved in some cases, but not others.

In contrast, the machine learning approach described herein, allows a full predictive model to be developed for the entire available chemical reaction space based on the autonomous navigation of the chemical space.

Raccuglia et al. describe methods for preparing metal oxides using machine-learning methods. However, the methods for preparing the metal oxides are not automated, and human-input is required in order to prepare each product. Whilst the authors look at generating a predictive model, and they use machine-learning to do so, there is no indication that the generation of this model might be placed under the control of an intelligent machine learning algorithm.

Ahneman et al. use machine-learning methods to predict the outcome of a particular class of organic reactions. The authors refer to the use of a high-throughput synthesiser and a robot in the generation of their training data, but there is no clear description of how the synthesiser and the robot are used. The robot itself is apparently used to analyse the reaction outcomes. The operation of the synthesiser is not described, and how this synthesiser selects reaction inputs is not disclosed. In the present invention, the control unit makes a sub-selection from amongst all the available inputs provided to the synthesiser. It seems that the synthesiser in Ahneman et al. is used to prepare all possible combinations of reactions from the available inputs (referred to as a full matrix). These results are then used to generate a predictive model for other, putative reactions.

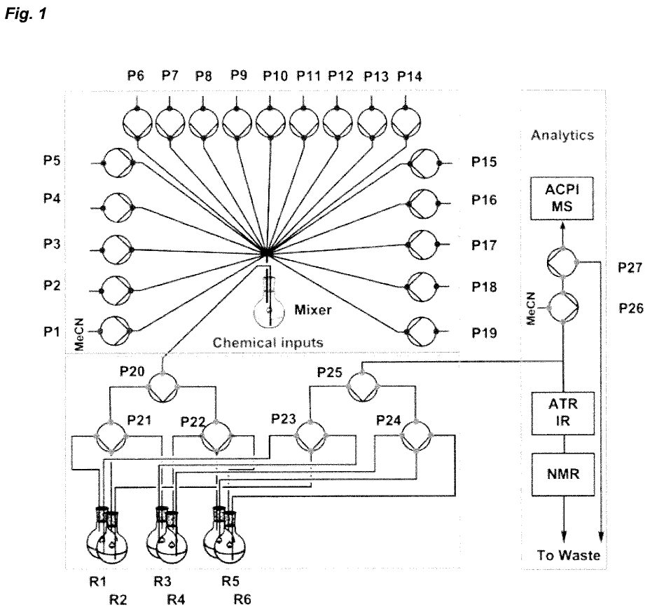

Dragone et al. focusses on identifying pathways within a multistep reaction that have the best reactivity (as shown in FIG. 1 of that work). This is more limited than the work described in the present case, which looks to predict the reactivity of all possible reactions within a reaction set, and it does not merely look for, and concentrate on, those combinations that are most reactive.

Points et al. considers the formation of a range of oil-in-water droplets. Here, a robot is used to generate the droplets and the physical properties of those droplets are analysed, with the analysis results used to generate a predictive model for the physical behaviour. However, Points et al. only look at the physical assembly of droplets, and the authors do not investigate chemical reactivity, as required by the methods of the present case.

Yoshida et al. looks to optimise the antibacterial activity of short 13-mer peptides. Here a range of variant peptides are prepared and analysed, and this analysis allows a fitness matrix to be generated, which records improvements in antimicrobial activity for the relevant mutations. From this matrix, the authors are able to generate a predictive model for the substitution of amino acids within the peptide.

As with Points et al., Yoshida et al. does not look to identify the outcome of a chemical reaction, nor predict chemical reactivity. Rather Yoshida et al. focusses on the optimisation of a property of a product, and the chemical and physical conditions that gives rise to that product are not investigated.

Although the peptides are generated by standard automated solid-phase synthesis, there is no suggestion that this is combined within a process where these are automatically analysed, and the results automatically feedback to inform later syntheses (as happens in the preferred methods in the present case).

Synthesiser

The system is provided with a synthesiser for performing reactions. The system is under the autonomous control of the control unit. Thus, the synthesiser can prepare reaction mixtures, and can subsequently dispose of reaction product mixtures upon completion of the reaction.

The synthesiser comprises one or more reaction spaces, each for the performance of a chemical or biological reaction. Where there are multiple reaction spaces, the synthesiser is capable of running reactions in each reaction space simultaneously or sequentially, and independently.

Particularly preferred synthesisers are fluidic synthesisers. Here, the synthesiser is adapted to control material transfer using fluids, typically liquid, to and from reaction spaces. Thus, reagents, solvent and catalysts may be provided, such as separately provided, in a fluid or as a fluid, and these fluids may be delivered to a reaction space for combination and reaction. After reaction, the fluid in the reaction space may be removed from that reaction space.

A reaction space may be provided by a traditional reactionware, such as a flask, or by other reactionware, such as reaction tubes, well plates and flow channels, amongst others. Thus, the reaction space, and the reactionware providing that space, is not particularly limited, and the skilled person will choose an appropriate reaction space for the reactions under investigation. Typically, the reactionware is one adapted for use with fluid transfer apparatus.

The reactionware may be provided together with apparatus for the delivery of material, such as reagents, catalysts and solvents, into the reactionware, and apparatus of the removal of material, such as the product reaction mixture, from the reactionware. Preferably, the synthesiser is provided with pumps, such as syringe pumps, for the delivery of reaction material into a reaction space.

The reactionware may also be provided together with apparatus for the manipulation of the reaction mixture. Thus, stirrers or mixers may be used in combination with the reactionware, optionally also with heaters, coolers, or light sources. Other standard reaction apparatus may also be provided, as might be required for the reaction set under investigation.

In the methods of the invention, a reactionware may be reused during the generation of a predictive model. Thus, it is not necessary to provide an individual reactionware for each reaction to be performed in the generation of the predictive model. As required, the contents of the reaction space in the reactionware may be emptied at reaction completion, optionally cleaned, and then made available for further use for another reaction.

The synthesiser is compatible with the analytical unit, to allow the analytical unit to analyse the reaction, for example during the reaction, or at deemed completion.

The synthesiser may allow the analytical unit to periodically extract samples from the reaction mixture for analysis.

Analytical Unit

The system comprises an analytical unit for analysing a reaction and the reaction products. The analytical unit comprises one or more analytical devices, or sensors, each for measuring a chemical or physical property of a reaction or a reaction species, such as a reagent, intermediate or product.

The analytical data collected by the one or more analytical devices is transmitted to the control unit for analysis.

The analytical device selected for the analytical unit is chosen based on the characteristic or characteristics of the reaction that are used to generate a predictive model.

It is preferred that the analytical unit is provided with multiple analytical devices for measuring different characteristics of the reaction or the reaction products. Such may allow the system to more accurately define a reaction outcome, and may also allow the system to more thoroughly analyse the reaction and the reaction products, with a greater opportunity to identify a change in reaction outcome with a change in a chemical and/or physical input. Accordingly this may assist in the generation of the predictive model.

By way of example, the analytical unit may comprise a one more analytical devices selected from a mass spectrometer, an NMR spectrometer, an IR spectrometer, UV and/or visible light spectrometer, including a colour sensor or a luminosity (luminance) sensor, pressure sensor, temperature sensor, and electrochemical sensor, amongst others. An analytical device may be used in combination with a chromatographic device for the at least partial separation of reaction components for analysis, such as a HPLC device.

In one embodiment, the analytical unit comprises a plurality of analytical devices.

In one embodiment, the analytical unit comprises one or more analytical devices selected from a mass spectrometer, an IR spectrometer, and an NMR spectrometer.

The analytical devices for use in the analytical unit are preferably those that are capable of providing analytical information in real time to the control unit. In this way the reaction outcome of a reaction may be rapidly determined by the control until from the analytical data, and this reaction outcome may be rapidly incorporated into the developing predictive model. Additionally, the control until may itself respond rapidly to the generated data and the determined reaction outcome, and it may select future inputs for subsequent reactions based on those reaction outcomes. Thus, the system may be rapidly responsive to the recorded reaction outcomes, and the reactions for performance may be revised appropriately to generate the predictive model rapidly.

The analytical data generated by the analytical devices in the analytical unit is transferred to the control unit for interpretation and storage.

Control Unit

The system is provided with a control unit. The control unit controls the synthesiser, and allows the synthesiser to perform chemical reactions within a reaction set without direct input from a user. The control until also receives analytical data from the analytical unit. The control unit analyses such data, and uses this date to generate a predictive model for the reactions in the reaction set.

The control unit is a computer that is suitably programmed to operate the synthesiser and to analyse analytical data. As described in further details,

The control unit is provided with a machine learning algorithm for the interpretation of analytical data, and for the generation of a predictive model based on that data, which is associated with the chemical and physical inputs used by the synthesiser.

The machine learning algorithm may be an artificial intelligence-based algorithm. The control unit may be programmed with a LDA algorithm for the interrogation of data, and the generation of the predictive model. The control unit may be programmed with a neural network algorithm, which may be used as an alternative to the LDA algorithm.

The control unit is provided with a database, or has access to a remote database, for the storage of analytical data together with the associated reaction conditions that gave rise to such data.

The control unit is provided with a user interface to allow the display of reaction information to the user, including the display of analytical data.

In some embodiments, the control until may operate the synthesiser semi-autonomously. Thus, a user may be permitted to instruct changes to the synthesiser as deemed necessary. For example, a user may deselect particular inputs for use in the synthesis. The user may do so if a particular input, and its associated reaction outcomes, is deemed of unworthy of exploration or use.

Alternatively, the user can choose to give greater prominence to certain chemical inputs in the subset of reactions that is performed within the reaction set. The user may choose to do so where that chemical input has a particular important, such as commercial importance.

Methods and Uses

The present invention provides for the use of a system for generating a predictive model for a reaction set. The methods of the invention are for generating and optionally validating a predictive model.

Here, a reaction set is the sum of the reaction outcomes for a plurality of chemical inputs, optionally together with physical inputs, into the system. Thus the reaction set contemplates all possible combinations of the chemical and physical inputs into the system. The reaction set may be regarded as describing the available reaction space which is defined by the chemical and physical inputs.

The methods of the invention are typically suitable for developing predictive models for reaction sets that comprise at least 100 different reactions, such as at least 500 different reactions, such as least 1,000 different reactions, such as 5,000 different reactions.

The methods of the present case are suitable for studying and predicting reaction outcomes for chemical and biological reactions within the reaction set. Here, there is no particular restriction on the types of reaction under investigation, nor is there any particular restriction on the reagents or products. Preferably, the methods and systems of the present case are for developing predictive models for chemical reactions, such as those for organic synthesis.

The methods of the present case are for use in studying and predicting reaction outcomes for a reaction set. Here, a reaction set is the sum of the reaction outcomes for a given series of chemical inputs, optionally together with physical inputs, for one or more reactions.

The methods of the invention may look at a reaction set where reactions within that set involve bond formation, such as covalent bond formation. For example, reactions within the reaction set may include carbon-carbon bond formation, carbon-oxygen bond formation, carbon-nitrogen bond formation, amongst others.

The methods of the invention may also be used to investigate a reaction set that includes changes in a physical state of a product, such as precipitation, crystallisation, and solubilisation, amongst others.

The references in the present case to the performance of a reaction, does not imply that a chemical reaction need occur, as the methods of the invention will also identify combinations of inputs that do not react. An accurate predictive model will therefore be based on reaction outcomes within a reaction subset that show no discernible changes over the inputs, as well as those reaction outcomes that are clearly associated with the formation of new products. As noted above, a reaction may also refer broadly to a change in the physical properties of a reaction mixture over time.

A reference to a chemical input in the present case is a reference to a reagent, catalyst or solvent, which may be provided for mixture with other reagents, catalysts or solvents. A reference to a physical input is a reference to heat, cool, light, ultrasound, or some other force, such as physical movement of the components in a reaction space, such as for mixing by stirring or shaking, or by flow.

In a simple embodiment, the reaction set is the sum of the reaction outcomes, where the chemical inputs are varied. The chemical inputs may be selected with a common reaction under consideration, for example a reaction having the same bond formation, or the same mechanism. For example, a reaction set may encompass an amide bond forming reaction, and the chemical inputs may provide for a combination of a plurality of amides and a plurality of carboxylic acids.

The methods of the present case do not assume and do not require a combination of chemical and physical inputs to lead to any reaction, nor to the formation of any particular product.

The control system, operating through the synthesiser, explores the available reaction space within a reaction set, to build a predictive model for all the reactions within the reaction set. Thus, the predictive model looks to predict the outcome of every combination of chemical, optionally together with physical, input within the reaction set, based on the performance of a fraction of the available reactions within the reaction set.

The control unit may halt the actions of synthesiser once it has obtained a degree of confidence in its ability to predict a reaction outcome. The confidence level may be set by the operator of the system.

Alternatively, the system may be suitably programmed to perform a set fraction of the reactions within the reaction set, and the control unit may halt the methods of synthesis once the relevant proportion of reactions is complete.

The methods of the invention look to develop a predictive model for a reaction. Here, the user has a choice as to the chemical or physical characteristic of a reaction that it is desirable to predict.

At its most general, the reaction outcome may simply be a difference in a physical of chemical characteristic between the initial reaction mixture and the reaction mixture at some point after the initial combination of chemical and physical inputs. Thus, the system may be used to explore whether any reaction happens at all for a particular combination. The use of binary encoding allows a matrix of reactions to be easily coded without the need for specific chemical information.

In a simple system, the reaction outcome may be reduced to a binary scoring of the reaction outcome as reactive or non-reactive, with necessarily any requirement for the system to identify any reaction products, or to quantify their relative or absolute amounts.

The binary scoring system may be applied to any chemical or physical feature that may be determined for a reaction mixture. Thus, whether a reaction is reactive or not may be replaced with simple interrogations of a reaction mixture relating to, for example, the presence or not of a spectroscopic signal within the analytical data. This spectroscopic signal may be associated with a certain functionality within a reagent or product, which is a signified of a certain reaction outcome, such as the consumption of a reagent or the formation of a particular product.

Further, the binary scoring system may be used to discern between reaction outcomes on the basis of a threshold value, against which a reaction may be scored depending upon whether the analytical data shows a characteristic exceeding the threshold value or not. The threshold value may be linked to a spectroscopic signal, for example, with the threshold set for a certain signal intensity, against the reaction outcome is judged. Other threshold values for use may relate to reaction yield, reaction temperature or reaction rate, amongst many others.

The reactions performed in the training set may alternatively be linked to a graded reaction outcome, which permits a spectrum of scoring options. Here, the predictive system is ultimately intended to provide a more nuanced prediction of reaction outcome compared with the binary system described above. This graded reaction system may similarly be related to the reaction outcomes described above, such as those linked to spectroscopic signals, with a greater range of options of describing those reaction outcomes in the scoring system.

The predictive system may then be used to develop a model for predicting whether a particular reaction will or will not have the characteristic under consideration in the binary system.

The reaction outcome for a reaction may be a physical or chemical property of a reaction product whose property is deemed important by the user to characterise the reaction, and is a useful property worthy of prediction. The analytical unit is provided to allow the reaction outcome to be determined, with reference to that particular property.

In some embodiments of the invention, with information from the analytical unit, the control unit may be used to identify the product of a reaction. The identification of products may be supported by knowledge of the chemical inputs involved, such as reagents, catalysts and solvents, and also the likely mechanisms involved.

A reaction outcome may be the identity of a product, optionally together with the yield of that product. Here, the predictive model may be used to predict the product of a reaction, optically together with its yield.

The control system may operate the synthesiser without knowledge of the chemical inputs into the reaction set. Thus, the control systems is permitted to operate blinded to the choice of chemical inputs, and without prejudice or expectation of any particular reactivity or reaction outcome. The control system is therefore permitted to select inputs as it sees fir to generate a reliable predictive model.

In practical terms, the chemical inputs, optionally together with the physical inputs, may be reduced to a vector representation of the available input pool in the form of a matrix. Here, each of the chemical and physical inputs is reduced to a simple coding within the matrix, such as to indicate presence or note, and the control until uses the vector representation to take generate a subset of reactions for developing the predictive model.

More particularly, the chemical and any physical inputs may be provided as binary options within a matrix, where a specific input may be coded as being present or absent. The reaction outcome, as determined, may then linked to a particular coding for the reaction.

The system is permitted to operate blind to the options that are provided for the chemical and physical inputs. Thus, the system need not necessarily be provided with the information about what the input is, or the machine learning for the purpose of the predictive model, may disregard the chemical and physical information that is provided to it. In this way, the machine is permitted to explore the available space purely in terms of the presence and absence of inputs. The system is accordingly not prejudiced and is not pre-conditioned to act in any way, or with any preference for a particular input or combination of inputs.

The control system may be permitted to randomly select reactions for performance as the initial stage for obtaining reaction outcomes for a subset of the reaction set. After performance of the reactions corresponding to this random selection, the control unit is permitted to choose future inputs, such as those where the control until believes will lead to the generation of a robust predictive model, or will allow the validation of the developing or developed model.

As noted previously, it is important for the control system to identify and perform reactions that are unreactive or lead to unwanted products, as this information of this type is important for the navigation of the available chemical space, and for the development of a robust predictive model.

The methods of the invention involve the generation of a collection of reaction outcomes for a subset of reactions within the reaction set. These reaction outcomes are held by the control unit and are interpreted by a machine learning algorithm in order to generate a predictive model.

In one embodiment, a collection of reaction outcomes, which is a subset of the reaction set, is subjected to a linear discriminant analysis (LDA), where the chemical inputs, optionally together with physical inputs, are linked to the reaction outcomes as target values. A neural network may also be used in place of the LDA, to generate the predictive model.

Reactions that are not within the subset, and are provided in the greater reaction set, and are therefore unperformed, may be subsequently scored based on a probability of reactivity predicted by the LDA model from the collection of reaction outcomes.

In a general aspect the present invention provides a method for generating a predictive model for a reaction set, where a reaction set is the sum of the reaction outcomes for a plurality of chemical inputs, optionally together with physical inputs, the method comprising the steps of:
(i) obtaining the reaction outcomes for a series of reactions, which series is a subset of the all the possible reaction outcomes for the reaction set;
(ii) considering the reaction outcomes in association with the chemical inputs, optionally together with the physical inputs, for each reaction; and
(iii) developing a predictive model for the reaction set from the reaction outcomes for the subset, such as based on an LDA or neural network model of the reaction outcomes.

In step (i), the reaction outcomes for a series of reactions may be obtain from published literature, such as journal articles, published patent specifications or other publically available sources.

The series of reactions for which there is reported reaction outcomes may comprise at least 10, such as at least 50, such as at least 100, such as at least 500, such as at least 1,000, such as at least 5,000 reactions.

Step (i) may comprise obtaining the reaction outcomes in part from published literature and in part by the performance of reactions within the subset. Here, the published literature may not report a sufficient number of reaction outcome to enable a user to develop a predictive model for the reaction set. Thus, a user may perform a number of reactions to obtain a sufficient number of reaction outcomes to develop the predictive model. Here, a user may use a system of the invention to generate the reaction outcomes from the relevant chemical inputs, optionally together with the physical inputs.

As described below, reaction outcomes may also be established by performance of all the reactions within a subset.

The present invention allows for the use of the system of the invention in a method to generate a predictive model. The method comprises the steps of:
(i) providing a system according to the invention, the system comprising a synthesiser for conducting reactions, which synthesiser is an automated synthesiser, an analytical unit for monitoring reactions performed by the synthesiser, and a control unit suitably programmed with a machine learning algorithm, for analysing analytical data from the analytical unit, and for controlling the synthesiser
(ii) making available to the synthesiser chemical inputs, optionally together with physical inputs;
(iii) permitting the synthesiser to perform a series of reactions using the available chemical inputs, optionally together with physical inputs, wherein the series is a subset of all the possible reactions for the combinations of the available chemical inputs, optionally together with physical inputs;
(iv) permitting the analytical unit to analyse each reaction, and allowing the analytical unit to transmit analytical data to the control unit;
(v) allowing the control unit to consider the analytical data to determine a reaction outcome for each reaction, and considering the reaction outcomes in association with the chemical inputs, optionally together with the physical inputs, for each reaction; and
(vi) developing a predictive model for the reaction set from the subset of reactions.

In the methods of the invention, as described above, the reaction outcomes for a subset of the reaction set are identified, such as determined from the literature and/or by performance of the reactions by a user, for example using a system of the invention.

The subset of reactions may be referred to as a training set. Here, the purpose of the reactions is to provide a base layer of reaction information for the system to analyse and to form the predictive model.

In one embodiment, the subset represents 50% or less of the available reactions within the reaction set, such as 40% or less, such as 30% or less, such as 20% or less, such as 10% or less.

The control unit may itself set the number of reactions for performance to generate the necessary training set. This number may vary as the system performs the reactions, with the control unit taking into account reaction outcomes that are widely varied across the reactions undertaken. Here, a larger subset may be required to generate the necessary predictive model for the widely variant reactions outcomes. Where the reactions outcomes are relatively conserved, showing little variance between the reactions undertaken, a smaller subset may be suitably used to generate the necessary predictive model.

The present case also provides methods for validating a predictive model, such as a model generated by the methods of the invention.

Also provided by the present case is a method of validating a predictive model, the method comprising the steps of:
(i) generating a predictive model for a reaction set according to a method of the invention, where the predictive model is generated from a subset of the all the reaction outcomes available within the reaction set,
(ii) selecting a reaction from the reaction set, where that reaction is not a reaction in the subset, and obtaining a predicted reaction outcome for the reaction from the predictive model; and
(iii) performing the reaction, establishing the reaction outcome, and comparing the reaction outcome against the predicted reaction outcome from the predictive model.

The method may further comprise the step of (iv) modifying the predictive model based on the reaction outcome of the reaction, for example where the reaction outcome differs from that predicted by the predictive model.

Step (ii) may comprise the selection of a plurality of reactions from the reaction set, where each reaction is not a reaction in the subset. Predicted outcomes may be obtained for each reaction, and these reactions may be performed according to step (iii) and the reaction outcomes compared against the predicted reaction outcomes.

The methods of the invention also provide for the identification of novel products and novel synthesis methods using the predictive models of the present case. The present case may be used to identify combinations of chemical inputs, optionally together with physical inputs, whose reaction outcome differs from that predicted by the predictive model. Such a reaction outcome may be associated with an unexpected, such as unpredicted, result.

In the methods of the invention, the control unit may develop a predictive model as it enclosures the available reaction space. This predictive model may be refined as the system performs further reactions, and generates further data, which allows the reaction outcome to be determined.

During that process of predictive model generation and refinement, the system may identify reactions that provide unpredicted reaction outcomes. The control unit can identify what combination of chemical inputs, optionally together with physical inputs, is associated with that reaction outcome. The control unit may then subsequently may then choose to explore the reaction space that is associated with one or more of the chemical inputs, optionally together with physical inputs, in order to generate a predictive model to account for the earlier unexpected reaction outcome.

In this wat, the system of the invention can identify unexpected reaction outcomes, and the system may be used to explore those input parameters giving rise to that unexpected outcome. The system may reveal to the user new reactivity and new products, and the system may allow the user to draw mechanistic insights into that reactivity and the products based on the predictive models, which can associate particular chemical inputs, optionally together with physical inputs, to that unexpected results.

In the methods of the invention, the control unit may order the synthesiser to repeat one or more reactions with the subset. The control until may do so to provide confirmation of a reaction outcome. The control until may do this where a particular reaction outcome is a departure from a predated outcome. The control unit may do this a part of a simple confirmation of a reaction result.

Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXPERIMENTAL

The following examples are provided solely to illustrate the present invention and are not intended to limit the scope of the invention, as described herein.

### General Experimental

Reagents were from Sigma Aldrich were used as received. Acetonitrile employed as a solvent in the platform was HPLC grade (VWR). Mass Spectra were recorded on a TOF MS (MicroTOF-Q MS) instrument equipped with an electrospray (ESI) source supplied by Bruker Daltonics Ltd. All analysis was collected in positive ion mode. The spectrometer was calibrated with the standard tune-mix to give a precision of ca. 1.5 ppm in the region of m/z 100-3000. NMR data was recorded on Bruker NMR Advance III 600 MHz or Bruker Avance 400 MHz. The spectra were recorded at 298 K using residual solvent proton peaks for scale reference e.g. (i. e. ¹H: δ (CDCl₃)=7.26; ¹³C: 6 (CDCl₃)=77.16). The chemical shifts are reported using δ-scale. All chemical shifts are reported in ppm and all coupling constants (J) are given in Hz. The following abbreviations were used to characterize spin multiplicities: s=singlet, d=doublet, t=triplet, q=quartet, m=multiplet, dd=double doublet, dt=double triplet, dq=double quartet, and ddt=double doublets of triplets. DEPT, COSY, HSQC, HMBC, and ROESY spectra were used for structure determination and structural assignments. New reaction candidates were analyzed using TLC chromatography and visualized using TLC plates with fluorescent indicator.

Syringe Pumps and Tubing

The control over the fluids was performed using with C3000 model, TriContinent™ pumps (Tricontinent Ltd, CA, USA) equipped with 5 mL syringes (TriContinent™) and four-way solenoid valve according to the requirements of the experiments. The pumps were connected using a RS232 port and a daisy-chain allowing connecting up to 16 pumps on a single RS232 bus. The commands to pumps were sent using pumps' proprietary control language, implemented in python module, allowing control over pumps, and error reporting functionality e.g. pumps malfunctioning. PTFE plastic tubing of ⅛ inch (3.175 mm) outer diameter was cut to specified length and connected using standard HPLC low pressure PTFE connectors and PEEK manifolds (supplied by Kinesis).

In-Line ATR-IR Spectroscopy

All spectra were recorded on a Thermo Scientific™ Nicolet™ iS™ 5 FT-IR equipped with a ZnSe Golden Gate Attenuated Total Reflectance Infrared (ATR-IR) flow cell. Frequencies are given in cm⁻¹. The resolution was set at 4 cm⁻¹ and each sample spectrum was recorded with 36 scans. The spectrometer was controlled by OMNIC software using python by ActiveX software framework. Before measurement of the spectra, the solvent (MeCN) was recorded as a background.

In-Line NMR Spectroscopy.

The NMR spectra were recorded using Spinsolve benchtop NMR from Magritek with a compact permanent magnet (43 MHz) based on Hallbach design, working on the lock-free basis (not requiring deuterated solvents). Shimming was performed using D₂O/H₂O mixture (9/1)(V/V) to minimize the half-width of the solvent peak. To realize the measurement of reaction mixtures, the spectrometer was equipped with in-home built flow cell with a standard 5 mm width to maximize sensitivity. The spectra were measured in a stopped-flow, by pumping reaction mixtures into the flow cell. The spectrometer was controlled by Spinsolve software by sending XML messages over a network connection.

Benchtop MS Spectroscopy

The spectra were recorded using Advion Expression using ACPI (atmospheric pressure) ionization technique. The detailed acquisition parameters can be found in ESI. The mass spectrometer was controlled using software python wrapper around AdvionAPI, allowing for complete control over instrument and acquisition parameters. The dilution of reaction mixtures necessary for recording the spectra of reaction mixtures was realized using two syringe pumps by diluting reaction mixtures 3125 times using solvent (MeCN) before measurements.

Flow Setup Implementation

The platform was assembled as presented in FIG. 1a using 27 syringe pumps, benchtop IR, NMR, and MS. Round bottom flasks (25 mL) were employed as mixer and reactors. 18 pumps were responsible for dispensing the chemicals to the mixer. Six pumps were used for moving the reaction mixture from mixer to proper reactor. One pump was assigned for pumping the solvent (MeCN). Two pumps were used to realize dilution step necessary for measurement of mass spectra.

The starting materials were prepared as 1.0 M solutions. Automatic data collecting, processing and control over platform was done in Python programming language Before the execution of the reaction the robot is cleaned three times, by flushing mixer, reactor flasks, and analytics. The reaction was performed by adding proper reagents to mixer (total volume 5.0 mL) in 1:1 ratio, transferring the reaction mixture to the reactor and saving reaction parameters: identity and volumes of starting materials. After two hours, the reaction mixture is transferred to the measurement loop, where the NMR and IR were recorded. The MS spectrum was recorded after dilution of the reaction mixture. After the reaction mixture has been measured, the mixer, reactor and analytics are cleaned by flushing them with solvent twice. The parallel execution of six reactions was implemented by shifting execution of each reaction in time so each experiment has access to liquid handling robot and analytics not colliding with other experiments. The spectra (NMR, IR) were also recorded for each chemical in the pool of starting materials (FIG. 4) used for calculation of theoretical spectrum of the reaction mixture.

In more detail, the system shown in FIG. 1(a) was set up according to the following protocol:

Solvent pump: Connect pump P1 input valve position to the bottle with solvent (acetonitrile) and output valve position to the mixer using needle and Luer to ¼″-28 Flat Bottom adapter (P1 on FIG. 1(a)).

Starting materials pumps: For each of eighteen pumps (P2-P19): Connect the input valve position of the pump to the bottle containing starting material with PTFE tubing. Connect the output valve position of the pump to the mixer flask equipped with septa using Luer to ¼″-28 Flat Bottom adapter and a long needle with PTFE tubing.

Reactors' pumps: 1. For pump (P20) (moving the reaction mixtures) connect the input valve position to the mixer using Luer to ¼″-28 Flat Bottom adapter and a needle (the needle should touch the bottom of the flask to ensure complete transfer of reaction mixtures. Connect the output and extra valve position of this pump (P20) to the next two pumps (P21-22) to the S valve position. Connect to the I, O, E positions of pumps P21-P22 to reactors R1-R6. For pumps, P23-P24 connect the I, O, E valve positions to the respective reactors. Connect the S valve positions of pumps 23 and 24 to the I, E and valve positions of the valve P25. Connect the output valve position of the pump P25 to a 3-way block connector. Analytics: Connect the ATR-IR flow cell to the 3-way block connector. Connect the second end of the ATR-IR flow cell to the NMR flow cell. Connect the output of the NMR flow cell S6 to the waste bottle.

Dilution pump and ms pump: Connect the input valve position of the pump P26 (equipped with 0.5 mL syringe) to the 3-way block connector. Connect the E valve position with the solvent bottle. Connect the O valve position with the S position of the pump P27. Connect the input valve of P27 to the Advion Mass spectrometer ACPI source. Connect the output valve position of P27 to the waste bottle. In total three RS232 connections were utilized to connect the pumps to the computer. The pumps can be conveniently connected to the USB via RS232 to USB converter cable.

Reaction Detection Algorithm Details

The reaction detection problem has been formulated as a binary classification problem given the spectra of the starting materials and reaction mixture (X) estimate the category of the reactivity Y=0 (unreactive) Y=1 (reactive). Training the SVM machine learning classifier for reaction prediction based on NMR/IR: The training set constituted 72 reactions. The category of reactivity has been assigned for each experiment by an expert chemist.

Processing of NMR spectra: a) Fourier transform of FID b) Auto phase the spectrum c) Reference the solvent d) Normalize the intensity of the solvent peak to 1.0 e) Cut the spectrum to the region between 2.5 and 12.0 ppm. The IR spectra were used without any pre-processing. 1. For each experiment in the training set: a) Load the nmr/ir spectrum of the reaction mixture b) Calculate the theoretical spectrum of the reaction mixture (sum of the starting materials) c) Read from file the expected reaction category y=0 (unreactive) y=1 (reactive) (the reactivity of the reactions has been labelled by an expert chemist) d) Combine theoretical and experimental spectra and add them to the training set. 2. Train SVM model on the reactivity labels (Y=0 or 1) and the spectra (X). The SVM classifiers for reaction detection were validate using leave one out cross validation achieving an accuracy of 86% percent for classifying the reaction as reactive or unreactive.

To automatically detect a chemical reaction by a robot the following algorithm has been implemented: 1. Load and process the NMR/IR spectrum of the reaction mixture; 2. Load reaction configuration file with volumes and concentration of the reagents; 3. Load and process the spectra of starting materials; 4. Create the theoretical spectrum of the reaction mixture by adding spectra of starting materials taking into account concentrations and volumes of each starting materials; 5. Feed the reaction mixture and theoretical IR/NMR spectrum to the trained SVM classifier; and 6. Predict reactivity of the reaction mixture y=0 (unreactive) or y=1 (reactive).

Autonomous Navigation of Chemical Space by the Robot.

The algorithm for exploration of chemical space starts by measuring 90 random experiments in the platform, and then each experiment in this set is processed to assess its reactivity and generate its representation. The ¹H NMR of the reaction mixture is auto processed by Fast Fourier Transform (FFT), phasing, and referencing the solvent peak. The intensity of the solvent peak was normalized to 1.0 (The solvent peak was used as an internal standard allowing easy addition of the spectra). The IR spectra were used without any preprocessing. Next, the theoretical spectra of the reaction mixture (being sum of the starting material) are constructed for NMR and IR. The spectra were normalized by removing the mean and scaled to unit variance. Reactivity of reaction mixture is assessed by feeding the NMR reaction mixture and NMR theoretical spectrum to SVM classifier (trained previously. The outcome of the classifier is y=0 (nonreactive) or y=1 (reactive). Similarly, the reactivity is assessed based on by SVM classifier based on IR spectra. An experiment would be classified as reactive if any of the above classifiers predicted it as reactive. The vector representation is generated utilizing the identity of the starting materials. The vector representation (X) and reactivity (Y) is added to the reaction database.

The machine learning algorithms were realized using sci-kit learn package in python (see Pedregosa et al.) After the initial the database of the reactions has been built, the LDA classifier is trained on the representation of the reactions (X) and their reactivity (Y). All the possible unperformed reactions are then scored by assigning them the probability of being reactive from LDA model. After the reactions with the highest score are done using the liquid handling robot, they are processed as described above updating the reaction database. Then, the LDA model is retrained on the updated database and robot iteratively explores chemical space until desired number of experiments is performed. The simulations of exploring the chemical space using above algorithm were performed on the data gathered by the robot.

The machine learning algorithm for exploration of chemical space was formulated as follows:

Start with a pool of unlabelled data (pool of chemical reactions from a given chemical space). Choose a few reactions at random and perform them in the system of the invention (measure reaction outcomes using in-line analytics) assign them to one of the two categories (reactive or unreactive) using an SVM reactivity classifier. For all the reaction performed, fit the LDA classifier—build the model of chemical space. The goal which was set to the robotic platform was to explore the most reactive parts of chemical space by querying the model to assess the reactivity of all unlabelled (unperformed) reactions and perform the most reactive reactions in the robot. Update the model of chemical space with all the reactions performed in the platform. Iteratively explore the chemical space searching for maximum reactivity;

In the initial phase, the chemical space is being randomly explored collecting the initial data required for building model of it. The robot performs the reaction by addition of starting materials to the mixer and then the reaction mixture is transferred to the proper reaction flask. After reaction time the reaction mixture is analysed with NMR and IR. The reaction is then encoded as a reaction vector X e.g. X=[1,1,0,0,0,0,0,0,0,0,0,0,0,0,0,0,1,0,0] and also is being classified as reactive or non-reactive Y=1 or Y=0 and this information is then added to the database performed reactions. Then the Linear Discriminant Analysis classifier is trained on the obtained data. All the unperformed reaction from the pool of the starting materials (two and three component) are then scored according to the probability of reaction obtained from the LDA. The reactions with the highest probability are then performed and analysed in the robotic system. Then the outcome of the reaction is added to the existing database of the reactions and the LDA classifier is retrained on the obtained data. In this way, it was possible to implement a feedback loop allowing the robot to explore the chemical space in iterative fashion gaining knowledge of chemical space as the chemical space is being explored. We were interested if this approach to the exploration of chemical space can lead to its efficient navigation and the robot can learn the reactivity patterns. The algorithm has been validated using 5-fold cross validation giving accuracy 86% of predicting reactivity and ROC area=0.89.

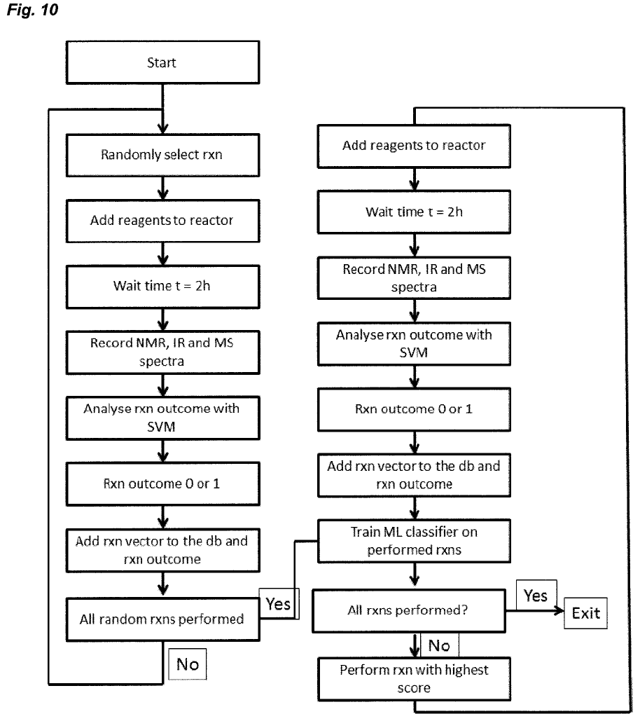

The detailed algorithm for the exploration of the chemical space is shown in FIG. 10.

Syntheses of Molecules Discovered by the Platform

The solutions of starting materials (1.0 M solutions in MeCN) were added to the round bottom flask (25 mL) in 1:1 ratio (total volume 5.0 mL) and stirred in room temperature for 2 hours. After this time the silica gel (4.0 g) was added and the solvent was evaporated. The products of the reaction were isolated using column chromatography. The syntheses of each compound were adjusted according to the need for each reaction.

Machine Learning Exploration of Suzuki-Miyaura Reaction

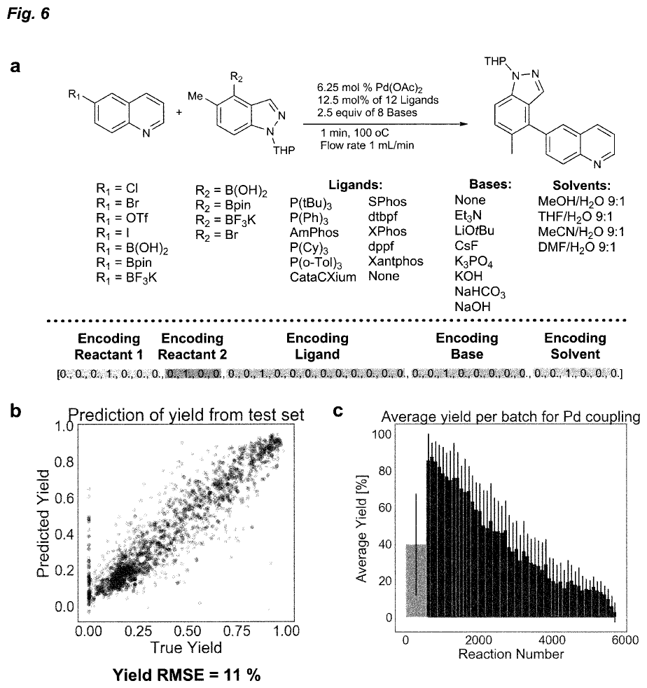

The reaction set described by Perera et al. contains 5,760 experiments of Suzuki-Miyaura coupling reaction, which are derived from the combinations of chemical inputs, here reagents, catalysts, bases and solvents, as shown in FIG. 6(a).

One-Hot Encoding of Reactions and Data Processing

Each reaction was one-hot encoded as a vector of length [1×37] (see FIG. 6(a)). This representation doesn't require any chemical knowledge about the chemical system being investigated. The yields were scaled to range 0.0-1.0. The constant parameters for all reactions were not encoded e.g. amount of palladium acetate, temperature, and flow rate.

Neural Network Architecture

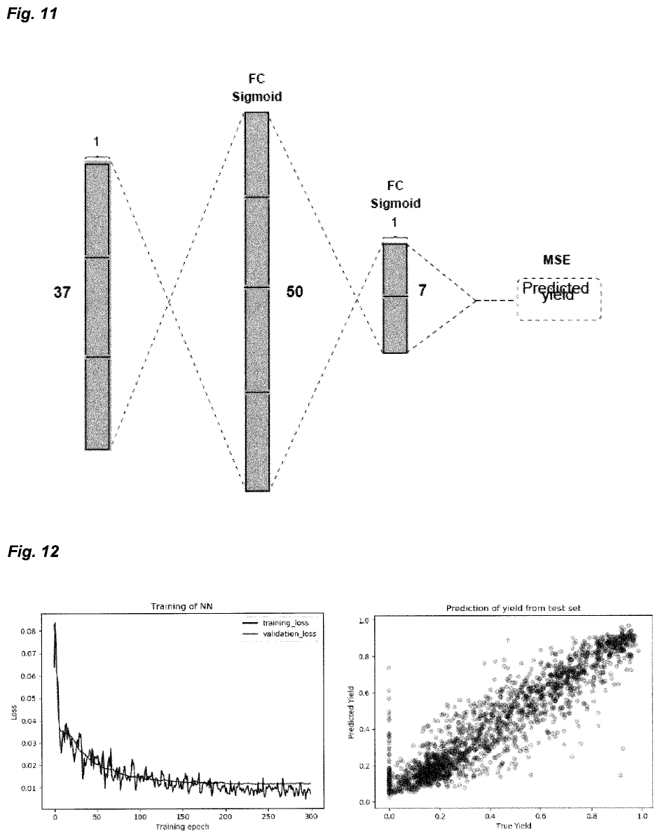

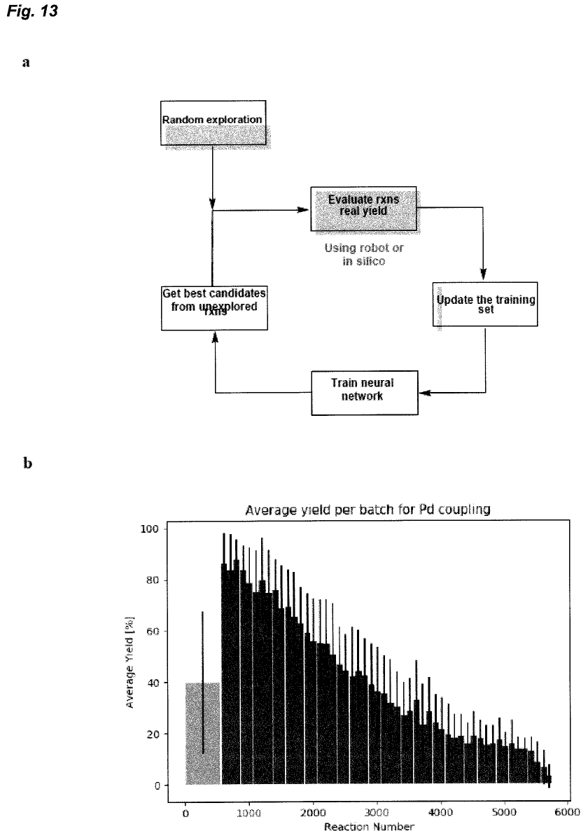

The neural network (NN) (see FIG. 11) comprised two layers: 50 neurons in the first fully connected layer with sigmoid activation function and dropout probability 0.8 for training. The second layer comprised 7 neurons in the fully connected layer also with sigmoid activation. The final prediction of yield was obtained as a linear regression of the output from the second layer. Mean squared error between predicted and experimental yield was implemented as a loss function to train NN. The NN was implemented in Tensorflow.

Validation of the Neural Network for Yield Prediction

The reaction data was randomly separation into training/validation/test sets at 60/10/30% respectively, and neural network was trained and validated using the training and validation data sets. The neural network was trained using Adam Optimizer (learning rate=0.005) and Mean Squared Error as a loss function with minibatch size of 100. To minimize overfitting the early stopping have been implemented. FIG. 12(a) shows the training process for 300 epochs. The mean squared error was MSE=0.01208 for 1728 reactions in the test set (see FIG. 12(b)).

Simulating Exploration of the Reaction Space Using Machine Learning

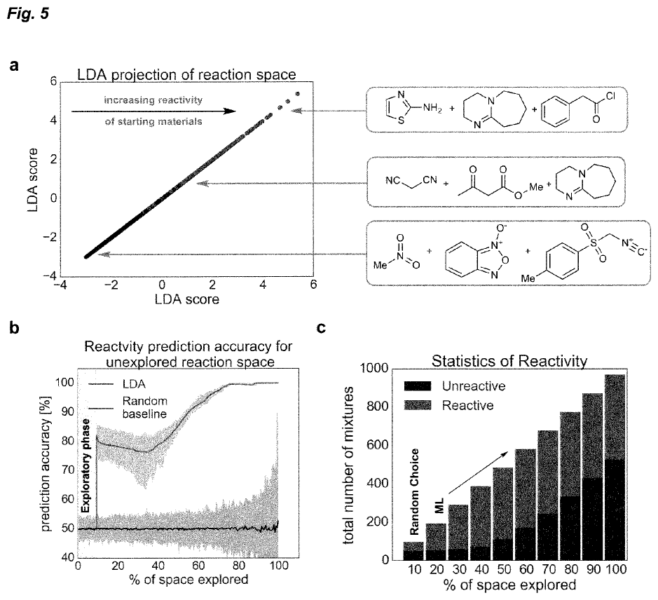

The main goal of the simulation was to show that the methods of the invention are able to help in design and development of organic reactions including high yielding transition metal catalysed transformations. Analogously to the simulation described above, the algorithm starts with random screening of chemical space by selecting 10% of available reactions (576 reactions), then the neural net is trained on this data and all the remaining reactions are then scored by the model. The candidates with the highest predicted yield are then added to the performed reactions, and the performance of the NN is evaluated by calculating the mean of true yield and standard deviation (SD) of the yield. The NN is then retrained and the whole cycle is repeated until the whole space is explored (see FIG. 5(a)). The space was explored in a batch of 100 to demonstrate compatibility with high throughput screening, as well as to evaluate the performance of NN. Exploratory phase consists of 576 reactions and gave on average yield about 39% with SD=27% (FIG. 5(b)). After training the NN, for the next batch of 100 reactions selected the average yield was typically 86% with SD=11%.

Commentary

The system of the present invention is exemplified by the chemical handling robot shown in FIG. 1, whose setup is described in detail above. This chemical handling robot comprises reactionware, an analytical unit for in-line spectroscopy and control unit for real-time data analysis and a feedback mechanism for control of the chemical handling in the reactionware.

The system robot was driven by a set of twenty-seven computer-controlled syringe pumps (Tricontinent C3000) responsible for liquid handling, dispensing chemicals, moving the reaction mixtures, and cleaning the reactionware after each reaction. Additionally, to increase the speed of exploration of reaction mixtures, the robot was configured such that six experiments could be executed in parallel at any one time, allowing up to 36 experiments to be performed per day.

To evaluate the outcome of a reaction the robot was equipped with real-time sensors: flow benchtop NMR (Spinsolve from Magritek; see Sans et al.), Mass Spec (Expression from Advion) and attenuated total reflection IR (Nicolet iS5 from Thermo Scientific; see Dragone et al.), recording the spectra of the reaction mixtures. The whole setup was controlled using software written in house using the Python programming language, and this software was responsible for controlling the pumps and the analytics, live-data processing, and implementation of the machine learning algorithms.

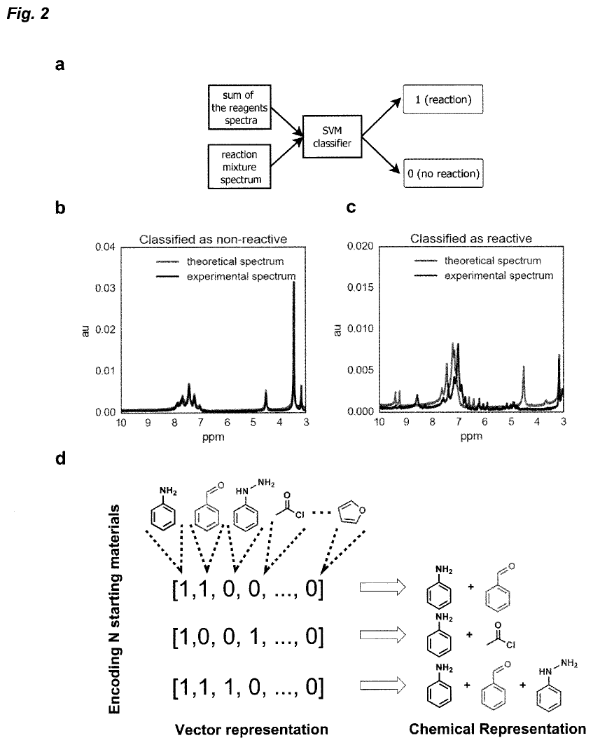

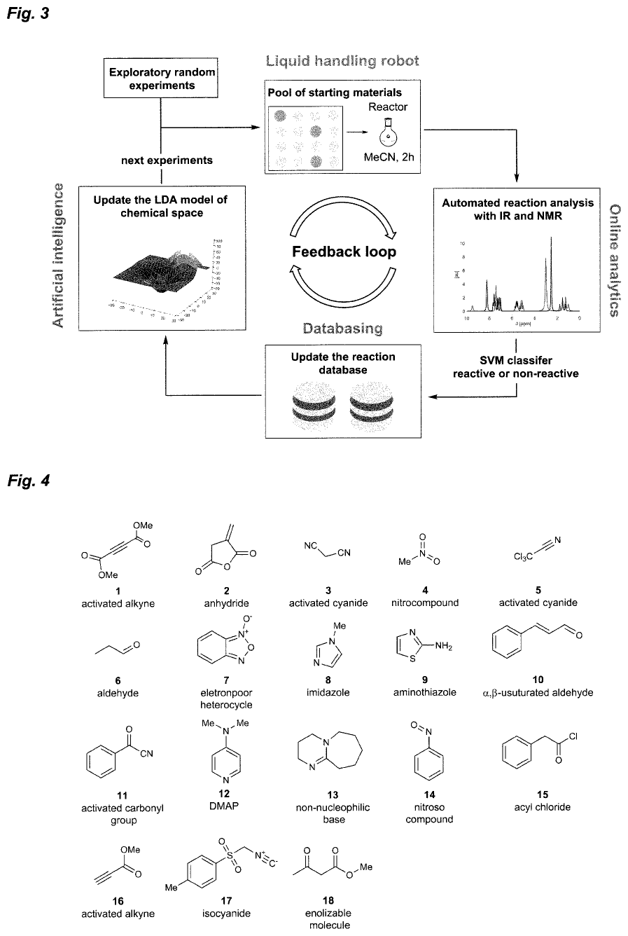

Initially, the algorithm was implemented in a way that would allow for automatic classification of the reaction mixtures as reactive or non-reactive which would be reported in binary form as zero or one. To achieve this goal, a Supported Vector Machine was built using a linear kernel model (FIG. 2(a)) (Cortes et al.), and this machine was used to compare a theoretical spectrum (this simply consists of the sum of the spectra of the starting materials) with a spectrum recorded from the robotic platform (NMR and IR), giving information about the reactivity of a given combination of starting materials.

When the theoretical spectrum was identical to the reaction mixture the algorithm would classify this reaction mixture as unreactive (FIG. 2(b)). On the other hand, when peaks of the starting materials were absent, and new peaks were found, the algorithm would classify a given combination of the starting materials as reactive (FIG. 2(c)). The model was trained on 72 reactive and unreactive mixtures, manually classified by an expert chemist, and could classify the reactivity of reaction mixtures with an accuracy of 86% as obtained from leave-one-out cross-validation. Even though mass spec analysis (ACPI-MS) was not useful for classification of reactivity, due to variable ionization affinities of starting materials and reaction mixtures, this analytical method was found to be useful for accelerating discovery by detecting high weight molecular ions.

The machine learning algorithm to explore chemical space needed an automatically generated representation of the reactions (Gómez-Bombarelli, R. et al.) As a representation of the data is key for machine learning (Bengio et al.), so it was hypothesised that a vector representation of chemical space might be useful for representing chemical reactions. To accomplish this, a reaction descriptor was created with a width corresponding to the number of starting materials in the pool of reagents and with those bits set to one for those reagents which were present for a given reaction, similarly to a one-hot encoding which is used in artificial intelligence for categorical data. FIG. 2(d) shows example vector representations for the model substrate pool consisting aniline, benzaldehyde, acetyl chloride, phenylhydrazine and furan.

To quantify the performance of the algorithm designed to explore chemical space, all possible 969 experiments formed from chemical space were recorded, as shown in FIG. 4. This allowed the running of multiple simulations exploring the patterns of reactivity using machine learning methods (see FIG. 5), as well as allowing the investigation of the influence of the random starting experiments on exploration of chemical space (the different starting conditions may lead to slightly different outcomes in exploring chemical space).

When Linear Discriminant Analysis (LDA) was performed on the data gathered, the algorithm was able to clearly differentiate between the reactive and non-reactive combinations of the starting materials. This means that the LDA can be useful for prediction of new reactivity, as shown in FIG. 5(a). The LDA model is also able to predict how reactive the given combination of starting materials is. Within FIG. 5(a), each blue dot represents a reaction which has been classified as unreactive (here, those dots having a low LDA scope), while the red dots show each reaction which has been classified as reactive (here, those dots having a high LDA score).

The position of the point on LDA plot reflects the reactivity of a given reaction mixture. Thus, points on the left side of the graph indicate the more unreactive starting materials, whilst points on the right-side result from reactive starting materials. In this way, the robot can learn the reactivity of the starting materials and efficiently navigate chemical space. For example, the reaction mixture composed from 2-aminothiazole (9), phenylacetyl chloride (15), and DBU (13), would be classified as highly reactive, a mixture of malononitrile (3), methylacetoacetate (18), and DBU (13), as moderately reactive. On the other hand, a mixture of nitromethane (4), benzofuroxan (7) and toluenesulfonylmethyl isocyanide (17) would be classified as unreactive. These assignments agree with basic chemical intuition which shows the predictive power of the model.

To further test the learning ability of the system of the invention, we simulations were performed showing the number of reactive vs non-reactive combinations of the starting materials chosen by the algorithm as the chemical space is being explored (FIG. 5(b)). In the initial stage, the space was randomly sampled, resulting in an equal number of reactive and non-reactive combinations being chosen by the algorithm. After reaching the desired number of reactions, the next decisions were made using LDA, which led to a rapid increase in the reactive number of the combination being chosen by the algorithm. i.e. by scanning 40% of the space the number of reactive combinations was doubled in comparison to the number of unreactive combinations. In the end, the algorithm identified that part of chemical space which was empty, thus, the last experiments which were chosen were unreactive.

The accuracy of predicting the reactivity is shown in FIG. 5(c). This figure shows that as the chemical space is progressively searched, the accuracy of the prediction of the reactivity increases along with the confidence intervals. This demonstrates that the robot can ‘self-learn’, exploiting this reactivity first approach. Additionally, the accuracy of LDA classifier for predicting the reactivity of reaction mixtures was validated using a five-fold cross validation giving an accuracy of 86+/−3%.

To explore the predictive power of the system, a recently described Suzuki-Miyaura reaction space (see FIG. 6(a)) was investigated by searching for reactions with the highest yield using the present machine learning machine learning approach (see Perera et al.)

To achieve this, a neural network was built as described above, and this used one-hot encoding to encode literature data for machine learning can be used for the prediction of yields. To do this, the data was partitioned into a training/validation/test set (3456/576/1728 reactions) to train and validate the neural network. When the neural network was tested it performed well giving yields with a RMSE=11% for 1728 reactions (see FIG. 6(b) for correlation between real yield and predicted yield).

Having established that this approach is able to predict the yield of Suzuki-Miyaura reaction, a simulation was performed to explore this chemical space analogously as described above for the robot here. Initially the algorithm started by randomly choosing 10% percent of the reaction space (576 reactions) and then the neural network was trained on this data. The unexplored parts of reaction space were then scored by the machine learning model and the next batch of candidates with best scores was selected and the true yield as evaluated.

The initial random guess had mean yield of 39% and standard deviation (SD) of 27% shown as a yellow bar in FIG. 6(c). The green bars show subsequent batches of 100 reactions chosen by ML. For example, the first batch of 100 reactions chosen by machine learning had mean yield of 85% and SD=14%. The subsequent batches contained less and less reactive starting materials, reaching finally unreactive parts of the reaction space. This was significant since it showed that only by doing 10% of the total number of reactions, it was possible to predict the outcomes of the remaining 90% without needing to physically do the experiments. Hence the machine learning approach of the invention can be used to explore a defined chemical space, in this case to design high yielding transformations and as well as search for reactivity, especially when coupled with high throughput experimentation methods.

Using the data from the real-time reactivity search robotic system, when the positions of the ¹H-NMR resonances show substantial changes in the chemical shifts different from starting materials, coupled with new peaks in the mass spectrum at high molecular weight, these were found to be highly predictive indicating that a novel transformation had occurred allowing to identify important reactivity hits as new discoveries of new reactions and molecules.

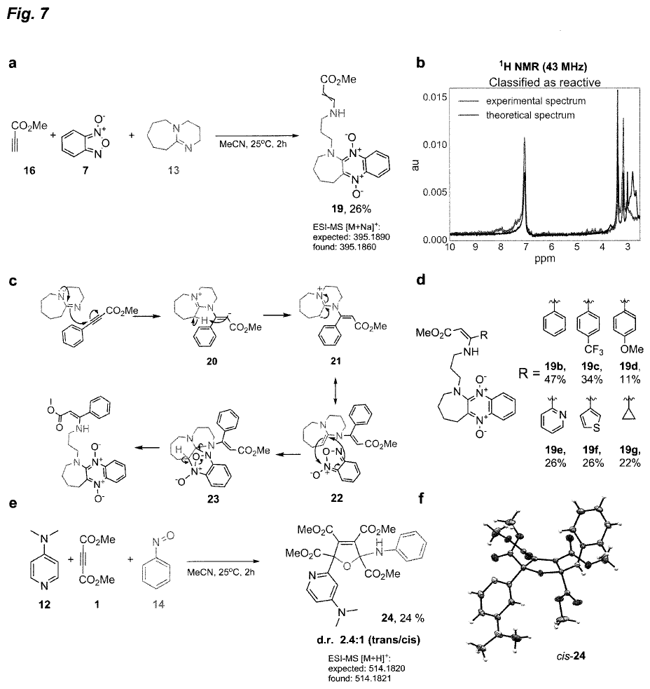

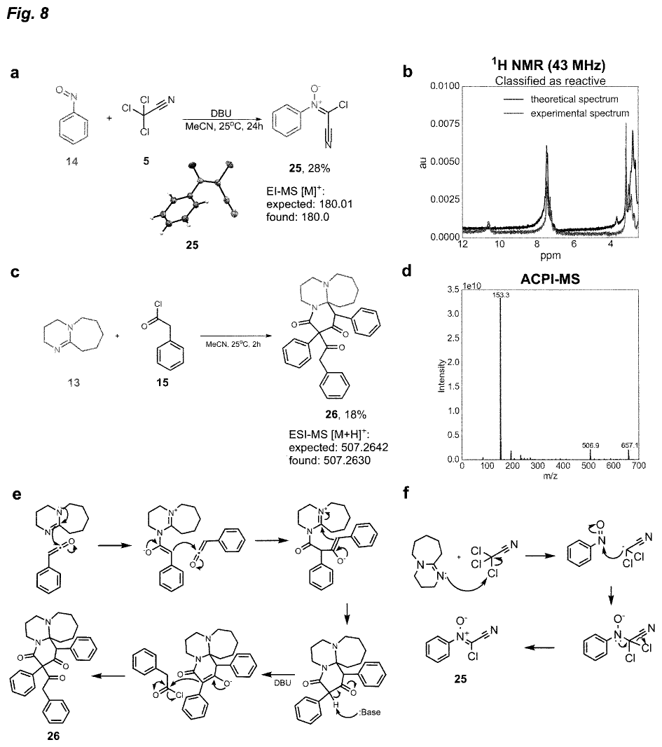

By working backwards from the reactive combinations, we used the conditions automatically discovered by the system to do reactions manually and isolate the products. For example, by analysing the spectra recorded by the robot we identified novel transformations (FIGS. 7 and 8).

For instance, analysis of the ¹H NMR spectrum recorded by the platform for the reaction between methyl propiolate (16) with benzofuroxan (7) and DBU (13) suggests an interesting transformation with new peaks visible in range δ=4.0-5.0 ppm and 7.9-8.5 ppm (FIG. 7(b)). FIG. 7(b) also shows the theoretical spectrum as being the sum of the starting materials. An attempt to isolate the reaction product gave a new molecule for which analysis of NMR spectra showed that it contained protons originating from all starting materials, and this suggested the compound results from a multicomponent reaction.

This observation was also confirmed by mass spectrometry by observing the molecular ion using ESI-MS [M+Na]⁺=395.1860. Analysis of the ¹H—¹³C HSQC and HMBC correlation spectra allowed for determination of its product structure 19; this confirms the observation that reaction between methyl propiolate (16) with benzofuroxan (7) and DBU (13) leads to a novel multicomponent reaction. FIG. 7(c) shows a suggested mechanism for this reaction.

In the first strep, DBU (13) undergoes a Michael addition to methyl propiolate (16) yielding the quaternary ammonium salt 20, after which the migration of the proton affords intermediate 21 for which another resonance structure is shown as structure 22. Compound 22 has enough dienophilic character to undergo a Diels-Alder reaction with benzofuroxan (7). Then, the [4+2] cycloadduct 23 rearranges, generating a N,N-pyrazine dioxide 19 as the final product.

Having established the structure of the product and the reaction mechanism, the utility of reaction was explored by synthesizing a small library of related molecules. By using substituted alkynes six new structurally diverse compounds were obtained in one step (19b-g). Furthermore, the multicomponent reaction of DMAD (1), nitrosobenzene (14) and DMAP (12) led to the formation of derivative of 2,5-dihydrofurane 24 with d.r. ratio (2.4:1) (trans/cis) (FIG. 7(e)), the structure of the product cis-24 was confirmed by single crystal X-ray diffraction.

Another interesting finding is presented in the FIG. 8(a). Chlorocyanonitrone 25-a member of an unreported class of nitrones—was isolated as the product of the reaction between trichloroacetonitrile (5) and nitrosobenzene (14) in the presence of DBU (13) (the structure of compound 25 was confirmed by X-ray analysis).

FIG. 8(b) presents the NMR spectrum recorded for this transformation by the system of the invention. New reactivity between ketenes and DBU (FIG. 8c) was also found, indicated by the peaks at high molecular weight recorded by the platform for this reaction (m/z=506.9 and m/z=657; see FIG. 8(d)). Under basic conditions, phenylacetyl chloride (15) is deprotonated by the DBU giving the phenyl ketene which undergoes a series of reactions with DBU giving the polycyclic azepine derivative 26 (see FIG. 8(e)), though DBU is usually considered to be a non-nucleophilic base. The suggested mechanisms for these transformations are presented in FIGS. 8(e) and 8(f).

To assess uniqueness of the reactions the Tanimoto similarity index was employed to compare the starting materials and products (see Bajusz et al.) To do this over 40 million reactions were considered, where these reactions were filtered by excluding the non-organic reactions, and then requiring the same number of reagents and product as the discoveries described herein, and finally by having all the required structural information.

With the filtering, this gave over ˜3.5 million reactions to compare. For each reaction the similarity between each reagent and the product was calculated, and then the mean established from the obtained values. For those reactions where the reagents underwent a slight modification to reach the product, this reaction similarity value would be close to 1.

Conversely, if the reagents change substantially so that the product is very different from the reagents, then the result would be close to 0.

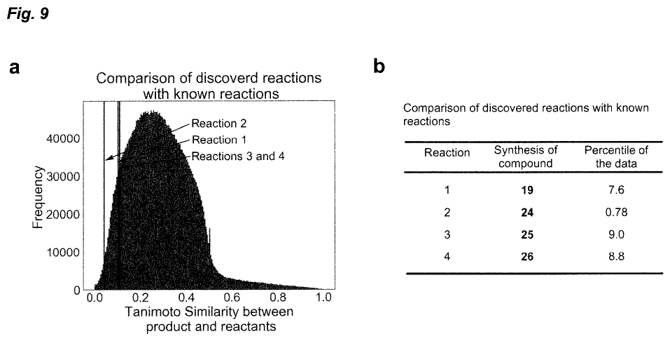

Looking at the histogram in FIG. 9) it can be seen that there is only one peak in the distribution and that the mean value is 0.29. All four of the reactions discovered here are more dissimilar than the mean; in fact all are in the top 10 percentile with reaction 2 which gives product 24 in the top 0.8 percentile (FIG. 9(b)). They are significantly more dissimilar than the expected reaction where it chosen at random.

The work presented here demonstrates that system provide with sensors such as NMR, IR, and MS, having a control until operating with machine learning can be used to autonomously search organic chemical space for new reactivity, leading to the discovery of reactions and molecules.

By exploiting machine learning algorithms, the system was able to navigate between reactive and non-reactive mixtures autonomously, learning reactivity patterns, and efficiently explore the most reactive regions of chemical space, requiring no prior knowledge.

Furthermore, after training, the system needs to perform only a fraction of possible the reactions to explore the most reactive/interesting parts of the chemical space. This leads to a much better performance than the random high throughput screening methods, saving time and resources. Additionally, the use of on-line analytics allows for convenient searching for novel transformations.

The simulation for the Suzuki-Miyaura reaction shows that the approach described herein can be easily expanded to search complex chemical systems including important transition metal catalyzed transformations, modifying more reactions parameters such as reaction catalysts, ligands, additives, temperature and solvent, which would maximize the capability of this system.

### REFERENCES

All documents mentioned in this specification are incorporated herein by reference in their entirety.
Ahneman et al. Science, 360, 186 (2018).
Allen et al. Chem. Rev. 113, 7287-7342, (2013).
Bajusz et al. J. Cheminform., 7, 20, (2015).
Bengio et al. IEEE Trans. Pattern Anal. Mach. Intell. 35, 1798-1828, (2013).
Cortes et al. Mach. Learn. 20, 273-297, (1995).
Dragone et al. Nat. Commun. 8, 15733, (2017).
Gil et al. Science 346, 171-172, (2014).
Gómez-Bombarelli et al. CoRR abs/1610.02415 (2016).
Graulich et al. Chem. Soc. Rev. 39, 1503-1512, (2010).
Naredla et al. Chem. Rev. 113, 6905-6948, (2013).
Palazzolo et al. PNAS, 114, 5564-5566, (2017).
Pedregosa et al. J. Mach. Learn. Res. 12, 2825-2830 (2011).
Perera et al. Science 359, 429-434, (2018).
Plata et al. J. Am. Chem. Soc. 137, 3811-3826, (2015).
Points et al. PNAS 118, 885, (2018).
Raccuglia et al. Nature 533, 73-76, (2016).
Reymond Acc. Chem. Res. 48, 722-730, (2015).
Sans et al. Chem. Sci. 6, 1258-1264, (2015).
Sans et al. Chem. Soc. Rev. 45, 2032-2043, (2016).
Warr Mol. Inform. 33, 469-476, (2014).
WO 2013/175240
Yoshida et al. Chem 4, 533, (2018).

1. A method to generate a predictive model for a reaction set, which reaction set is the sum of the reaction outcomes for a plurality of chemical inputs, optionally together with physical inputs, the method comprises the steps of:
(i) providing a system comprising a synthesiser for conducting reactions, which synthesiser is an automated synthesiser, an analytical unit for monitoring reactions performed by the synthesiser, and a control unit suitably programmed with a machine learning algorithm, for analysing analytical data from the analytical unit, and for controlling the synthesiser
(ii) making available to the synthesiser chemical inputs, optionally together with physical inputs;
(iii) permitting the synthesiser to perform a series of reactions using the available chemical inputs, optionally together with physical inputs, wherein the series is a subset of all the possible reactions for the combinations of the available chemical inputs, optionally together with physical inputs;
(iv) permitting the analytical unit to analyse each reaction, and allowing the analytical unit to transmit analytical data to the control unit;
(v) allowing the control unit to consider the analytical data to determine a reaction outcome for each reaction, and considering the reaction outcomes in association with the chemical inputs, optionally together with the physical inputs, for each reaction; and
(vi) developing a predictive model using the machine learning algorithm for the reaction set from the subset of reactions.

2. The method of claim 1, wherein the machine learning algorithm is a linear discriminant analysis algorithm or a neural network algorithm.

3. The method of claim 1, wherein the analytical unit comprises a plurality of analytical devices, where each device is for providing real time analytical data to the control unit.

4. The method of claim 1, wherein the analytical unit comprises a mass spectrometer, an IR spectrometer, and an NMR spectrometer.

5. The method of claim 1, wherein the control unit operates autonomously to control the selection of chemical inputs, optionally together with physical inputs, for the synthesiser and to generate the predictive model from the selected chemical inputs, optionally together with physical inputs.

6. The method of claim 1, wherein the chemical inputs, optionally together with physical inputs, are coded in a matrix form with binary classification for each chemical input, and optionally each physical input.

7. The method of claim 1, wherein the subset represents 30% or less of the available reactions within the reaction set.

8. The method of claim 1, wherein the number of available reactions within the reaction set is at least 500 reactions.

9. The method of claim 1, including the additional steps of validating the predictive model, the method comprising the additional steps of:
(a) selecting a reaction from the reaction set, where that reaction is not a reaction in the subset, and obtaining a predicted reaction outcome for the reaction from the predictive model; and
(b) performing the reaction, establishing the reaction outcome, and comparing the reaction outcome against the predicted reaction outcome from the predictive model.

10. The method of claim 9, which further comprises the step of (c) modifying the predictive model based on the reaction outcome of the reaction, where the reaction outcome differs from that predicted by the predictive model.

11. The method of claim 9, wherein the control unit identifies reaction outcomes that are not predicted or predictable, and the control unit identifies combination of chemical inputs, optionally together with physical inputs, that is associated with the unpredicted reaction outcome, thereby to identify new reactivity and/or new products within the available reaction set.

12. A method for generating a predictive model for a reaction set, which reaction set is the sum of the reaction outcomes for a plurality of chemical inputs, optionally together with physical inputs, the method comprising the steps of:
(i) obtaining the reaction outcomes for a series of reactions, which series is a subset of the all the possible reaction outcomes for the reaction set;
(ii) considering the reaction outcomes in association with the chemical inputs, optionally together with the physical inputs, for each reaction; and
(iii) developing a predictive model for the reaction set from the reaction outcomes for the subset.

13. The method of claim 12, wherein the reaction outcomes are obtained or obtainable from a publically available source, such as the published literature.

14. A system for generating a predictive model for a reaction set, the system comprising a synthesiser for conducting reactions, which synthesiser is an automated synthesiser, an analytical unit for monitoring reactions performed by the synthesiser, and a control unit suitably programmed with a machine learning algorithm, for analysing analytical data from the analytical unit, and for controlling the synthesiser.
